(19) United States
(12) Patent Application Publication
(10) Pub. No.: US 2007/0293476 A1
(43) Pub. Date: Dec. 20, 2007

(54) CO-THERAPY FOR THE TREATMENT OF EPILEPSY AND RELATED DISORDERS

(76) Inventors: Virginia L. Smith-Swintosky, Hatfield, PA (US); Michael H. Parker, Chalfont, PA (US); Allen B. Reitz, Lansdale, PA (US); Bruce E. Maryanoff, Forest Grove, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/750,579

(22) Filed: May 18, 2007

Related U.S. Application Data
(60) Provisional application No. 60/802,001, filed on May 19, 2006.

(51) Int. Cl. A61K 31/55; A61K 31/454; A61K 31/405; A61K 31/4025; A61K 31/381; A61K 31/343

(52) U.S. Cl. 514/217.03; 514/419; 514/443; 514/469; 514/217.08; 514/323; 514/422

(57) ABSTRACT

The present invention is directed to a method for the treatment of epilepsy and related disorders comprising administering to a subject in need thereof, co-therapy with a therapeutically effective amount of a benzo-heteroaryl sulfamide derivative as described herein and a therapeutically effective amount of one or more anticonvulsant and/or anti-epileptic agents.

### CROSS REFERENCE TO RELATED APPLICATIONS

This application claims the benefit of U.S. Provisional Application 60/802,001, filed on May 19, 2006, which is incorporated by reference herein in its entirety.

### FIELD OF THE INVENTION

The present invention is directed to a method for the treatment of epilepsy and related disorders comprising administering to a subject in need thereof, co-therapy with a therapeutically effective amount of a benzo-heteroaryl sulfamide derivative as described herein and a therapeutically effective amount of one or more anticonvulsant and/or anti-epileptic agents.

### BACKGROUND OF THE INVENTION

Epilepsy describes a condition in which a person has recurrent seizures due to a chronic, underlying process. Epilepsy refers to a clinical phenomenon rather than a single disease entity, since there are many forms and causes of epilepsy. Using a definition of epilepsy as two or more unprovoked seizures, the incidence of epilepsy is estimated at approximately 0.3 to 0.5 percent in different populations throughout the world, with the prevalence of epilepsy estimated at 5 to 10 people per 1000.

An essential step in the evaluation and management of a patient with a seizure is to determine the type of seizure that has occurred. The main characteristic that distinguishes the different categories of seizures is whether the seizure activity is partial (synonymous with focal) or generalized.

Partial seizures are those in which the seizure activity is restricted to discrete areas of the cerebral cortex. If consciousness is fully preserved during the seizure, the clinical manifestations are considered relatively simple and the seizure is termed a simple-partial seizure. If consciousness is impaired, the seizure is termed a complex-partial seizure. An important additional subgroup comprises those seizures that begin as partial seizures and then spread diffusely throughout the cortex, which are known as partial seizures with secondary generalization.

Generalized seizures involve diffuse regions of the brain simultaneously in a bilaterally symmetric fashion. Absence or petit mal seizures are characterized by sudden, brief lapses of consciousness without loss of postural control. Atypical absence seizures typically include a longer duration in the lapse of consciousness, less abrupt onset and cessation, and more obvious motor signs that may include focal or lateralizing features. Generalized Tonic-clonic or grand mal seizures, the main type of generalized seizures, are characterized by abrupt onset, without warning. The initial phase of the seizure is usually tonic contraction of muscles, impaired respiration, a marked enhancement of sympathetic tone leading to increased heart rate, blood pressure, and pupillary size. After 10-20 s, the tonic phase of the seizure typically evolves into the clonic phase, produced by the superimposition of periods of muscle relaxation on the tonic muscle contraction. The periods of relaxation progressively increase until the end of the ictal phase, which usually lasts no more than 1 min. The postictal phase is characterized by unresponsiveness, muscular flaccidity, and excessive salivation that can cause stridorous breathing and partial airway obstruction. Atonic seizures are characterized by sudden loss of postural muscle tone lasting 1-2 s. Consciousness is briefly impaired, but there is usually no postictal confusion. Myoclonic seizures are characterized by a sudden and brief muscle contraction that may involve one part of the body or the entire body.

There remains a need to provide an effective treatment for epilepsy and related disorders.

### SUMMARY OF THE INVENTION

The present invention is directed to a method for the treatment of epilepsy and related disorders comprising administering to a subject in need thereof co-therapy with a therapeutically effective amount of one or more anticonvulsant or anti-epileptic agents and a therapeutically effective amount of a compound of formula (I) 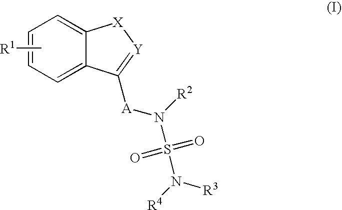
wherein
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to three additional heteroatoms independently selected from the group consisting of O, N and S;
or a pharmaceutically acceptable salt thereof.

### DETAILED DESCRIPTION OF THE INVENTION

The present invention is directed to a method for the treatment of epilepsy and related disorders comprising administering to a subject in need thereof, co-therapy with a therapeutically effective amount of one or more anticonvulsant or anti-epileptic agents and a therapeutically effective amount of a compound of formula (I) 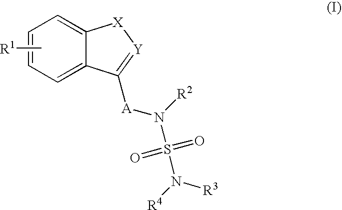
or a pharmaceutically acceptable salt thereof, wherein R¹, R², R³, R⁴, —X—Y— and A are as herein defined.

In an embodiment, the present invention is directed to a method for the treatment of epilepsy and related disorders comprising administering to a subject in need thereof, co-therapy with a therapeutically effective amount of a one or more anticonvulsant or anti-epileptic agents and a therapeutically effective amount of a compound of formula (I) 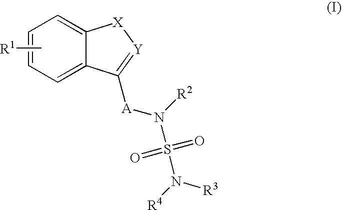
wherein
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and methyl;
alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

In an embodiment, the present invention is directed to a method for the treatment of epilepsy and related disorders comprising administering to a subject in need thereof, co-therapy with a therapeutically effective amount of a one or more anticonvulsant or anti-epileptic agents and a therapeutically effective amount of a compound of formula (I) wherein
R¹ is selected from the group consisting of hydrogen and halogen;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and methyl;
and pharmaceutically acceptable salts thereof.

In an embodiment, the present invention is directed to a method for the treatment of epilepsy and related disorders comprising administering to a subject in need thereof, co-therapy with a therapeutically effective amount of a one or more anticonvulsant or anti-epileptic agents and a therapeutically effective amount of a compound of formula (I) wherein
R¹ is selected from the group consisting of hydrogen and halogen; wherein the halogen is bound at the 4-, 5- or 7-position;
X—Y is selected from the groups consisting of —O—CH—, —O—C(CH₃)—, —S—CH—, —S—C(CH₃)—, —N(CH₃)—CH— and —CH═CH—CH—;
A is selected from the group consisting of —CH₂— and —CH(CH₃)—;
R² is hydrogen;
R³ and R⁴ are each hydrogen;
and pharmaceutically acceptable salts thereof.

In an embodiment, the present invention is directed to a method for the treatment of epilepsy and related disorders comprising administering to a subject in need thereof, co-therapy with a therapeutically effective amount of a one or more anticonvulsant or anti-epileptic agents and a therapeutically effective amount of a compound of formula (I) wherein
R¹ is hydrogen;
X—Y is selected from the groups consisting of —O—CH—, —O—C(CH₃)—, —S—CH—, —S—C(CH₃)—, —N(CH₃)—CH— and —CH═CH—CH—;
A is selected from the group consisting of —CH₂— and —CH(CH₃)—;
R² is hydrogen;
R³ and R⁴ are each hydrogen;
and pharmaceutically acceptable salts thereof.

In an embodiment, the present invention is directed to a method for the treatment of epilepsy and related disorders comprising administering to a subject in need thereof, co-therapy with a therapeutically effective amount of a one or more anticonvulsant or anti-epileptic agents and a therapeutically effective amount of a compound of formula (I) wherein
R¹ is selected from the group consisting of hydrogen halogen, hydroxy, methoxy, trifluoromethyl, nitro and cyano; preferably, R¹ is selected from the group consisting of hydrogen and halogen; more preferably, R¹ is selected from the group consisting of hydrogen and halogen, wherein the halogen is bound at the 4-, 5- or 7-position;
X—Y is —S—CH—;
A is selected from the group consisting of —CH₂— and —CH(CH₃)—;
R² is selected from the group consisting of hydrogen and methyl; preferably, R² is hydrogen;
R³ and R⁴ are each independently selected from the group consisting of hydrogen and halogen; preferably, R³ and R⁴ are each hydrogen;
and pharmaceutically acceptable salts thereof.

In an embodiment of the present invention R¹ is selected from the group consisting of hydrogen, chloro, fluoro and bromo. In another embodiment of the present invention, the R¹ group is other than hydrogen and bound at the 4-, 5- or 7-position, preferably at the 5-position. In yet another embodiment of the present invention, the R¹ group is other than hydrogen and bound at the 5-, 6- or 8-position, preferably at the 6-position. In yet another embodiment of the present invention, R¹ is selected from the group consisting of hydrogen and halogen. In yet another embodiment of the present invention, R¹ is selected from the group consisting of hydroxy and methoxy. In yet another embodiment of the present invention, R¹ is selected from the group consisting of hydrogen, halogen and trifluoromethyl. In yet another embodiment of the present invention, R¹ is selected from the group consisting of hydrogen, halogen, trifluoromethyl, cyano and nitro. In yet another embodiment of the present invention, R¹ is selected from the group consisting of hydrogen, halogen, trifluoromethyl and cyano. In yet another embodiment of the present invention, R¹ is selected from the group consisting of trifluoromethyl and cyano. In yet another embodiment of the present invention, R1 is selected from the group consisting of hydrogen, 4-bromo, 5-chloro, 5-fluoro, 5-bromo, 5-trifluoromethyl-5-cyano and 7-cyano.

In an embodiment of the present invention R² is hydrogen. In another embodiment of the present invention R³ and R⁴ are each hydrogen. In yet another embodiment of the present invention R² is hydrogen, R³ is hydrogen and R⁴ is hydrogen.

In an embodiment of the present invention, R³ and R⁴ are each independently selected from the group consisting of hydrogen and C₁₋₄alkyl. In another embodiment of the present invention, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S.

In an embodiment of the present invention, R³ and R⁴ are each independently selected from the group consisting of hydrogen, methyl and ethyl. In another embodiment of the present invention, R³ and R⁴ are each independently selected from the group consisting of hydrogen and methyl. In yet another embodiment of the present invention, R³ and R⁴ are each independently selected from the group consisting of hydrogen and ethyl. In yet another embodiment of the present invention, R³ is hydrogen and R⁴ is ethyl.

In an embodiment of the present invention R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, S and N. In another embodiment of the present invention R³ and R⁴ are taken together with the nitrogen atom to which they are bound to form a 5 to 7 membered saturated ring structure, optionally containing one to two additional heteroatoms independently selected from the group consisting of O, S and N. In another embodiment of the present invention R³ and R⁴ are taken together with the nitrogen atom to which they are bound to form a 5 to 7 membered aromatic ring structure, optionally containing one to two additional heteroatoms independently selected from the group consisting of O, S and N.

Preferably, R³ and R⁴ are taken together with the nitrogen atom to which they are bound to form a 5 to 6 membered saturated, partially unsaturated or aromatic ring structure, optionally containing one to two additional heteroatoms independently selected from the group consisting of O, S and N. More preferably, R³ and R⁴ are taken together with the nitrogen atom to which they are bound to form a 6 membered saturated, partially unsaturated or aromatic ring structure, optionally containing one to two additional heteroatoms independently selected from the group consisting of O, S and N.

Preferably, R³ and R⁴ are taken together with the nitrogen atom to which they are bound to form a 5 to 7 (more preferably 5 to 6) membered saturated or aromatic ring structure, optionally containing one to two (preferably one) additional heteroatoms independently selected from the group consisting of O, S and N (preferably O or N, more preferably N).

In another embodiment of the present invention, R³ and R⁴ are taken together with the nitrogen atom to which they are bound to form a 5 to 6 membered saturated or aromatic ring structure, optionally containing one to two (preferably one) additional heteroatoms independently selected from the group consisting of O, S and N (preferably O or N, more preferably, N).

Preferably, the 5 to 7 membered saturated, partially unsaturated or aromatic ring structure contains 0 to 1 additional heteroatoms independently selected from the group consisting of O, S and N. Preferably, the heteroatom is independently selected from the group consisting of O and N, more preferably, the heteroatom is N.

Suitable examples of the 5 to 7 membered, saturated, partially unsaturated or aromatic ring structures which optionally contain one to two additional heteroatoms independently selected from the group consisting of O, S and N include, but are not limited to pyrrolyl, pyrrolidinyl, pyrrolinyl, morpholinyl, piperidinyl, piperazinyl, imidazolyl, pyrazolyl, pyridyl, imidazolyl, thiomorpholinyl, pyrazinyl, triazinyl, azepinyl, and the like. Preferred 5 to 7 membered, saturated, partially unsaturated or aromatic ring structures which optional containing one to two additional heteroatoms independently selected from the group consisting of O, S and N include, but are not limited, to imidazolyl, pyrrolidinyl, piperidinyl and morpholinyl.

In an embodiment of the present invention A is —CH₂—.

In an embodiment of the present invention X—Y is selected from the group consisting of —S—CH—, —O—CH—, —O—C(CH₃)—, —N(CH₃)—CH— and —CH═CH—CH—. In another embodiment of the present invention X—Y is selected from the group consisting of —S—CH—, —O—CH—, —O—C(CH₃)— and —CH═CH—CH—. In yet another embodiment of the present invention X—Y is selected form the group consisting of —S—CH—, —O—CH—, —O—C(CH₃)— and —N(CH₃)—CH—. In yet another embodiment of the present invention X—Y is selected from the group consisting of —S—CH—, —O—CH—, —N(CH₃)—CH— and —CH═CH—CH—. In yet another embodiment of the present invention X—Y is selected from the group consisting of —S—CH—, —O—CH— and —CH═CH—C—. In yet another embodiment of the present invention, X—Y is selected from the group consisting of —S—CH— and —O—CH—. In yet another embodiment of the present invention, X—Y is selected from the group consisting of S—CH—, —S—C(CH₃)—, —O—CH—, —O—C(CH₃)— and —N(CH₃)—CH—.

In an embodiment of the present invention, X— is —S—CH—. In another embodiment of the present invention X—Y is —CH═CH═CH—. In yet another embodiment of the present invention X—Y is —N(CH₃)—CH—. In yet another embodiment of the present invention X—Y is selected from the group consisting of —O—CH— and —O—C(CH₃)—.

In an embodiment, the present invention is directed to a compounds selected from the group consisting of N-(benzo[b]thien-3-ylmethyl)-sulfamide; N-[(5-chlorobenzo[b]thien-3-yl)methyl]-sulfamide; N-(3-benzofuranylmethyl)-sulfamide; N-[(5-fluorobenzo[b]thien-3-yl)methyl]-sulfamide; N-(1-benzo[b]thien-3-ylethyl)-sulfamide; N-(1-naphthalenylmethyl)-sulfamide; N-[(2-methyl-3-benzofuranyl)methyl]-sulfamide; N-[(5-bromobenzo[b]thien-3-yl )methyl]-sulfamide; N-[(4-bromobenzo[b]thien-3-yl)methyl]-sulfamide; N-[(7-fluorobenzo[b]thien-3-yl)methyl]-sulfamide; N-[(1-methyl-1H-indol-3-yl)methyl]-sulfamide; N-[(4-trifluoromethylbenzo[b]thien-3-yl )methyl]-sulfamide; N-[(4-cyanobenzo[b]thien-3-yl)methyl]-sulfamide; N-[(benzo[b]thien-3-yl)methyl]-sulfamoylpyrrolidine; N-[(benzo[b]thien-3-yl)methyl]-N′-ethylsulfamide; Imidazole-1-sulfonic acid [(benzo[b]thien-3-yl)methyl]-amide; and pharmaceutically acceptable salts thereof.

In an embodiment, the present invention is directed to a method for the treatment of epilepsy and related disorders comprising administering to a subject in need thereof a therapeutically effective amount of one or more anticonvulsant and/or anti-epileptic agents with a compound of formula (I), wherein the compound of formula (I) is N-(benzo[b]thien-3-ylmethyl)-sulfamide or a pharmaceutically acceptable salt thereof.

In another embodiment, the present invention is directed to a method for the treatment of epilepsy and related disorders comprising administering to a subject in need thereof a therapeutically effective amount of one or more anticonvulsant and/or anti-epileptic agents with a compound of formula (I), wherein the compound of formula (I) is N-[(5-fluorobenzo[b]thien-3-yl)methyl]-sulfamide or a pharmaceutically acceptable salt thereof.

Additional embodiments of the present invention, include those wherein the substituents selected for one or more of the variables defined herein (i.e. R¹, R², R³, R⁴, X—Y and A) are independently selected to be any individual substituent or any subset of substituents selected from the complete list as defined herein.

Representative compounds useful in the methods of the present invention are as listed in Table 1 and 2, below.

TABLE 1
Representative Compounds of Formula (I)

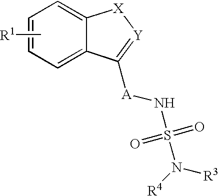

| ID No. | R¹ | —X—Y— | A | R³ | R⁴ |
|---|---|---|---|---|---|
| I | H | —S—CH— | —CH₂— | H | H |
| 3 | 5-Cl | —S—CH— | —CH₂— | H | H |
| 6 | H | —O—CH— | —CH₂— | H | H |
| 7 | H | —N(CH₃)—CH— | —CH₂— | H | H |
| 8 | 5-F | —S—CH— | —CH₂— | H | H |
| 9 | H | —S—CH— | —CH(CH₃)— | H | H |
| 10 | H | —CH═CH—CH— | —CH₂— | H | H |
| 13 | H | —O—O(CH₃) | —CH₂— | H | H |
| 15 | 5-Br | —S—CH— | —CH₂— | H | H |
| 17 | 4-Br | —S—CH— | —CH₂— | H | H |
| 18 | 7-F | —S—CH— | —CH₂— | H | H |
| 19 | 5-CF₃ | —S—CH— | —CH₂— | H | H |
| 20 | 5-CN | —S—CH— | —CH₂— | H | H |
| 21 | H | —S—CH— | —CH₂— | H | ethyl |

TABLE 2

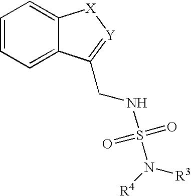

| ID No. | —X—Y— | R3 + R4 together with the N atom |
|---|---|---|
| 101 | —S—CH— | N-pyrrolidinyl |
| 102 | —S—CH— | N-imidazolyl |

As used herein, “halogen” shall mean chlorine, bromine, fluorine and iodine.

As used herein, the term “alkyl” whether used alone or as part of a substituent group, include straight and branched chains. For example, alkyl radicals include methyl, ethyl, propyl, isopropyl, butyl, isobutyl, sec-butyl, t-butyl, pentyl and the like. Unless otherwise noted, “C₁₋₄alkyl” means a carbon chain composition of 1-4 carbon atoms.

When a particular group is “substituted” (e.g., alkyl, phenyl, aryl, heteroalkyl, heteroaryl), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

Unless otherwise noted, the position at which the R¹ substituent is bound will be determined by counting around the core structure in a clockwise manner beginning at the X—Y positions as 1,2 and continuing from thereon as follows:

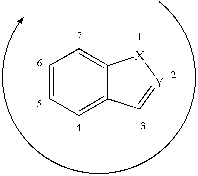

Should the X—Y substituent be —CH═CH—CH—, then the X—Y group will be counted as 1, 2, 3 and counting then continued clockwise around the core structure as previously noted.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

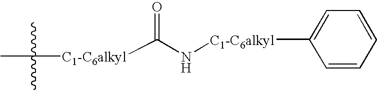

Abbreviations used in the specification, particularly the Schemes and Examples, are as follows:
DCE=Dichloroethane
DCM=Dichloromethane
DMF=N,N-Dimethylformamide
DMSO=Dimethylsulfoxide
LAH=Lithium Aluminum Hydride
MTBE=Methyl-tert-butyl ether
THF=Tetrahydrofuran
TLC=Thin Layer Chromatography

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following:
acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following:
acids including acetic acid, 2,2-dichlorolactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and
bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

As used herein, unless otherwise noted, the terms “epilepsy and related disorders” or “epilepsy or related disorder” shall mean any disorder in which a subject (preferably a human adult, child or infant) experiences one or more seizures and/or tremors. Suitable examples include, but are not limited to, epilepsy (including, but not limited to, localization-related epilepsies, generalized epilepsies, epilepsies with both generalized and local seizures, and the like), seizures associated with Lennox-Gastaut syndrome, seizures as a complication of a disease or condition (such as seizures associated with encephalopathy, phenylketonuria, juvenile Gaucher's disease, Lundborg's progressive myoclonic epilepsy, stroke, head trauma, stress, hormonal changes, drug use or withdrawal, alcohol use or withdrawal, sleep deprivation, fever, infection, and the like), essential tremor, restless limb syndrome, and the like. Preferably, the disorder is selected from epilepsy (regardless of type, underlying cause or origin), essential tremor or restless limb syndrome, more preferably, the disorder is epilepsy (regardless of type, underlying cause or origin) or essential tremor.

The term “subject” as used herein, refers to an animal, preferably a mammal, most preferably a human adult, child or infant, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of one or more of the symptoms of the disease or disorder being treated; and/or reduction of the severity of one or more of the symptoms of the disease or disorder being treated.

Wherein the present invention is directed to co-therapy or combination therapy, comprising administration of one or more compound(s) of formula (I) and one or more anticonvulsant or anti-epileptic agents, therapeutically effective amount shall mean that amount of the combination of agents taken together so that the combined effect elicits the desired biological or medicinal response. For example, the therapeutically effective amount of co-therapy comprising administration of a compound of formula (I) and at least one suitable anti-epileptic agent would be the amount of the compound of formula (I) and the amount of the suitable anti-epileptic agent that when taken together or sequentially have a combined effect that is therapeutically effective. Further, it will be recognized by one skilled in the art that in the case of co-therapy with a therapeutically effective amount, as in the example above, the amount of the compound of formula (I) and/or the amount of the suitable anti-epileptic agent individually may or may not be therapeutically effective.

As used herein, the terms “co-therapy” and “combination therapy” shall mean treatment of a subject in need thereof by administering one or more anticonvulsant and/or anti-epileptic agent(s) and one or more compounds of formula (I), wherein the compound(s) of formula (I) and the anticonvulsant and/or anti-epileptic agent(s) are administered by any suitable means, simultaneously, sequentially, separately or in a single pharmaceutical formulation. Where the compound(s) of formula (I) and the anticonvulsant and/or anti-epileptic agent(s) are administered in separate dosage forms, the number of dosages administered per day for each compound may be the same or different. The compound(s) of formula (I) and the anticonvulsant and/or anti-epileptic agent(s) may be administered via the same or different routes of administration. Examples of suitable methods of administration include, but are not limited to, oral, intravenous (iv), intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The compound(s) of formula (I) and the anticonvulsant and/or anti-epileptic agent(s) may be administered according to simultaneous or alternating regimens, at the same or different times during the course of the therapy, concurrently in divided or single forms.

As used herein, unless otherwise noted, the term “antiepileptic agent” and the abbreviation “AED” will be used interchangeably with the term “anti-convulsant agent,” and as used herein, refer to an agent capable of treating, inhibiting or preventing seizure activity or ictogenesis when the agent is administered to a subject or patient.

Suitable examples of anti-convulsant and/or anti-epileptic agents include, but are not limited to:
(a) AMPA antagonists such as AMP-397, E-2007, NS-1209, talampanel, and the like;
(b) Benzodiazepines such as diazepam, lorazepam, clonazepam, clobazam, and the like;
(c) Barbiturates such as phenobarbital, amobarbital, methylphenobarbital, primidone, and the like;
(d) Valproates such as valproic acid, valproate semisodium, valpromide, and the like;
(e) GABA agents such as gabapentin, pregabalin, vigabatrin, losigamone, retigabine, rufinamide, SPD-421 (DP-VPA), T-2000, XP-13512, and the like;
(f) Iminostilbenes such as carbamazepine, oxcarbazepine, and the like;
(g) Hydantoins such as phenytoin sodium, mephenytoin, fosphenytoin sodium, and the like;
(h) NMDA antagonists such as harkoseramide, and the like;
(i) Sodium channel blockers such as BIA-2093, CO-102862, lamotrigine, and the like;
(j) Succinimides such as methsuximide, ethosuximide, and the like; and
(k) AEDS such as acetazolamide, clomthiazole edisilate, zonisamide, felbamate, topiramate, tiagabine, levetiracetam, briveracetam, GSK-362115, GSK-406725, ICA-69673, CBD cannabis derivative, isovaleramide (NPS-1776), RWJ-333369, safinamide, seletracetam, soretolide, stiripentol, valrocemide, and the like.

In an embodiment, the anti-convulsant and/or anti-epileptic agent is selected from the group consisting of brivaracetam, carbamazepine, clobazam, clonazepam, ethosuximide, felbamate, gabapentin, lacosamide, lamotrigine, levetiracetam, oxcarbazepine, phenobarbital, phenytoin, pregabalin, primidone, retigabine, rufinamide, safinamide, seletracetam, talampanel, tiagabine, topiramate, valproate, vigabatrin, zonisamide, benzodiazepines, barbiturates and sedative hypnotics.

In another embodiment, the anti-convulsant and/or anti-epileptic agent(s) is selected from the group consisting of of carbamazepine, clobazam, clonazepam, ethosuximide, felbamate, gabapentin, lamotrigine, levetiracetam, oxcarbazepine, phenobarbital, phenytoin, pregabalin, primidone, retigabine, rufinamide, talampanel, tiagabine, topiramate, valproate, vigabatrin and zonisamide.

In another embodiment, the anti-convulsant and/or anti-epileptic agent(s) is selected from the group consisting of carbamazepine, lamotrigine, phenobarbital, phenytoin, topiramate, valproate and zonisamide. Preferably, the anti-convulsant and/or anti-epileptic agent(s) is selected from the group consisting of carbamazepine, gabapentin, lamotrigine, levetiracetam, oxcarbazepine, phenytoin, pregabalin, valproate and topiramate. More preferably, the anti-convulsant and/or anti-epileptic is selected from the group consisting of gabapentic, lamotrigine, levetiracetam, valproate and topiramate.

In an embodiment, the present invention is directed to a method for the treatment of epilepsy and related disorders comprising administering to a subject in need thereof co-therapy with a therapeutically effective amount of one or more compounds (I) as described herein and a therapeutically effective amount of one or more of the compounds as disclosed in US Patent Publication 2006 0041008 A1, which is herein incorporated by reference in its entirety.

In another embodiment, the present invention is directed to a method for the treatment of epilepsy and related disorders comprising administering to a subject in need thereof co-therapy with a therapeutically effective amount of one or more compounds (I) as described herein and a therapeutically effective amount of one or more of the compounds as disclosed in US Patent Publication 2006 0282887 A1, which is herein incorporated by reference in its entirety.

As used herein, the term “composition” is intended to encompass a product comprising the specified ingredients in the specified amounts, as well as any product which results, directly or indirectly, from combinations of the specified ingredients in the specified amounts.

Compounds of formula (I) wherein A is —CH₂— may be prepared according to the process outlined in Scheme 1.

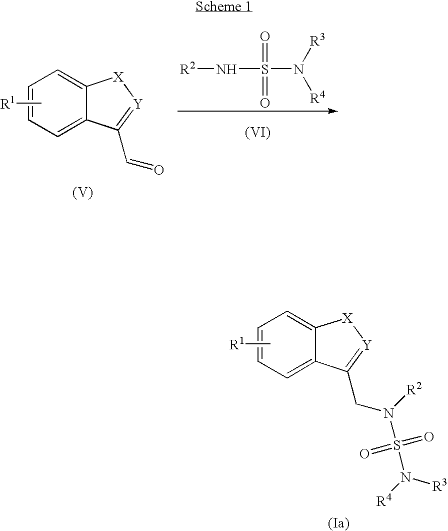

Accordingly, a suitably substituted compound of formula (V), a known compound or compound prepared by known methods, is reacted with a suitably substituted compound of formula (VI), a known compound or compound prepared by known methods, wherein the compound of formula (VI) is present in an amount in the range of about 2 to about 5 equivalents, in an organic solvent such as ethanol, methanol, dioxane, and the like, preferably, in an anhydrous organic solvent, preferably, at an elevated temperature in the range of about 50° C. to about 100° C., more preferably at about reflux temperature, to yield the corresponding compound of formula (Ia).

Compounds of formula (I) may alternatively be prepared according to the process outlined in Scheme 2.

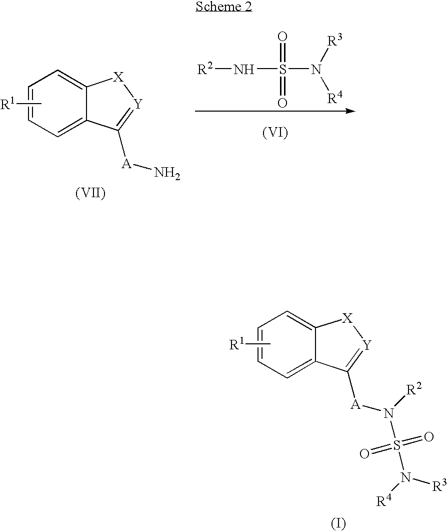

Accordingly, a suitably substituted compound of formula (VII), a known compound or compound prepared by known methods, is reacted with a suitably substituted compound of formula (VI), a known compound or compound prepared by known methods, wherein the compound of formula (VI) is present in an amount in the range of about 2 to about 5 equivalents, in an organic solvent such as THF, dioxane, and the like, preferably, in an anhydrous organic solvent, preferably, at an elevated temperature in the range of about 50° C. to about 100° C., more preferably at about reflux temperature, to yield the corresponding compound of formula (I).

Compounds of formula (VII) wherein A is —CH₂— may, for example, be prepared by according to the process outlined in Scheme 3.

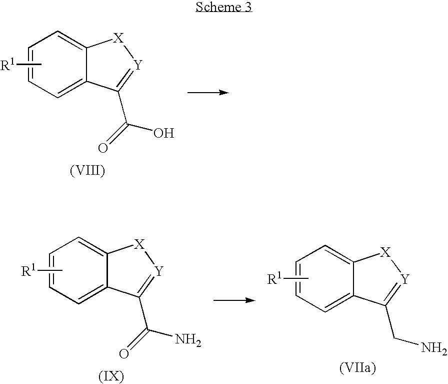

Accordingly, a suitably substituted a compound of formula (VIII), a known compound or compound prepared by known methods is reacted with an activating agent such as oxalyl chloride, sulfonyl chloride, and the like, and then reacted with an amine source such as ammonia, ammonium hydroxide, and the like, in an organic solvent such as THF, diethyl ether, DCM, DCE, and the like, to yield the corresponding compound of formula (IX).

The compound of formula (IX) is reacted with a suitably selected reducing agent such as LAH, borane, and the like, in an organic solvent such as THF, diethyl ether, and the like, to yield the corresponding compound of formula (VIIa).

Compounds of formula (VII) wherein A is —CH(CH₃)— may, for example, be prepared according to the process outlined in Scheme 4.

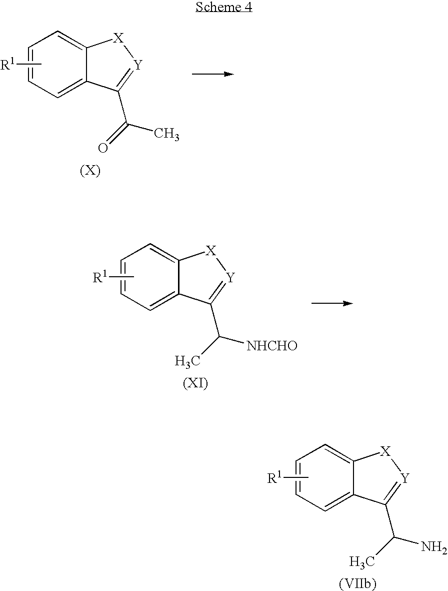

Accordingly, a suitably substituted compounds of formula (X), a known compound or compound prepared by known methods, is reacted with a mixture of formamide and formic acid, wherein the mixture of formamide and formic acid is present in an amount greater than about 1 equivalent, preferably, in an excess amount of greater than about 5 equivalent, at an elevated temperature of about 150° C., to yield the corresponding compound of formula (XI).

The compound of formula (XI) is hydrolyzed by reacting with concentrated HCl, concentrated H₂SO₄, and the like, at an elevated temperature, preferably at reflux temperature, to yield the corresponding compound of formula (VIIb).

Compounds of formula (VII) may alternatively, be prepared according to the process outlined in Scheme 5.

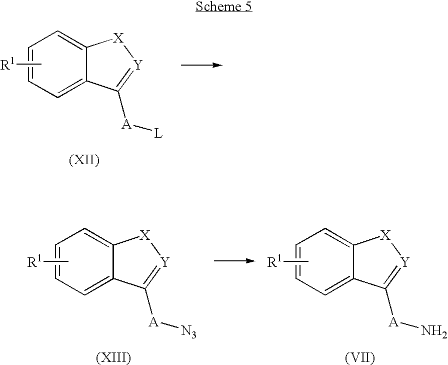

Accordingly, a suitably substituted compound of formula (XII), wherein L is a leaving group such as Br, Cl, I, tosylate, mesylate, and the like, a known compound or compound prepared by known methods, is reacted with sodium azide, in an organic solvent such a DMF, DMSO, methanol, ethanol, and the like, to yield the corresponding compound of formula (XIII).

The compound of formula (XIII) is reacted with a suitably selected reducing agent such as LAH, triphenylphosphine, H_{2(g)}, and the like, according to known methods, to yield the corresponding compound of formula (VII).

Compounds of formula (VII) wherein A is CH₂ and X—Y is —O—CH₂— may, for example, be prepared according to the process outlined in Scheme 6.

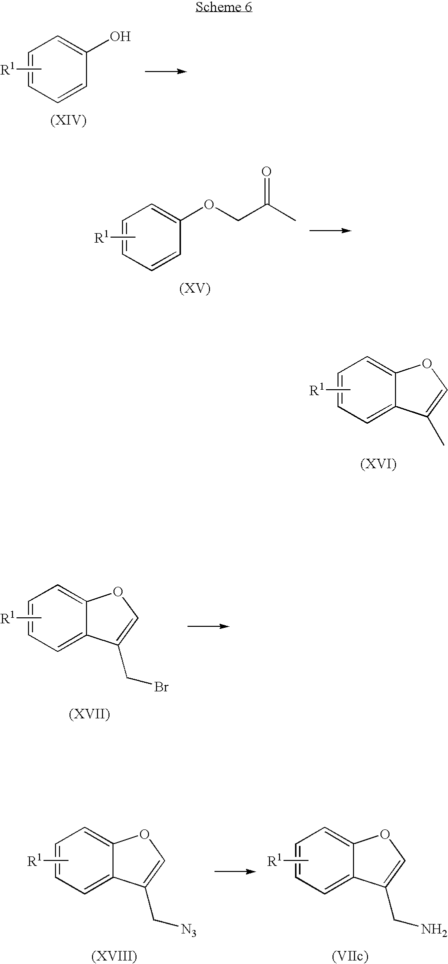

Accordingly, a suitably substituted phenol, a compound of formula (XIV), a known compound or compound prepared by known methods is reacted with bromoacetone, a known compound, in the presence of a base such as K₂CO₃, Na₂CO₃, NaH, triethylamine, pyridine, and the like, in an organic solvent such as acetonitrile, DMF, THF, and the like, optionally at an elevated temperature, to yield the corresponding compound of formula (XV).

The compound of formula (XV) is reacted with an acid such as polyphosphoric acid, sulfuric acid, hydrochloric acid, and the like, preferably with polyphosphoric acid, preferably in the absence of a solvent (one skilled in the art will recognize that the polyphosphoric acid acts as the solvent), to yield the corresponding compound of formula (XVI).

The compound of formula (XVI) is reacted with a source of bromine such as N-bromosuccinimide in the presence of benzoylperoixde, Br₂, and the like, in an organic solvent such as carbon tetrachloride, chloroform, DCM, and the like, preferably in a halogenated organic solvent, to yield the corresponding compound of formula (XVII).

The compound of formula (XVII) is reacted with sodium azide, in an organic solvent such a DMF, DMSO, methanol, ethanol, and the like, to yield the corresponding compound of formula (XVIII).

The compound of formula (XVI II) is reacted with a suitably selected reducing agent such as LAH, triphenylphosphine, H_{2(g)}, and the like, according to known methods, to yield the corresponding compound of formula (VIIc).

Compounds of formula (V) wherein X—Y is —S—CH— may, for example, be prepared according to the process outlined in Scheme 7.

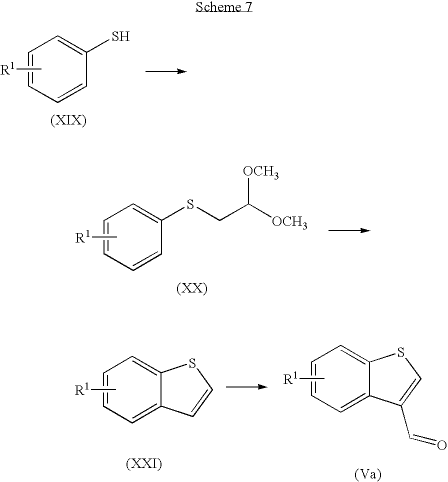

Accordingly, a suitably substituted compound of formula (XIX), a known compound or compound prepared by known methods is reacted with choroacetaldehyde dimethyl acetal or bromoacetaldehyde dimethyl acetal, a known compound, in the presence of a base such as potassium-tert-butoxide, sodium-tert-butxide, potassium carbonate, potassium hydroxide, and the like, in an organic solvent such as THF, DMF, acetonitrile, and the like, to yield the corresponding compound of formula (XX).

The compound of formula (XX) is reacted with reacted with an acid such as polyphosphoric acid, sulfuric acid, hydrochloric acid, and the like, preferably with polyphosphoric acid in the presence of chlorobenzene, preferably in the absence of a solvent (one skilled in the art will recognize that the polyphosphoric acid and/or the chlorobenzene may act as the solvent), at an elevated temperature in the range of from about 100 to 200° C., preferably at an elevated temperature of about reflux temperature, to yield the corresponding compound of formula (XXI).

The compound of formula (XXI) is reacted with a formylating reagent such as dichloromethyl methyl ether, and the like, in the presence of Lewis acid catalyst such as titanium tetrachloride, aluminum trichloride, tin tetrachloride, and the like, in an organic solvent such as DCM, chloroform, and the like, at a temperature in the range of from about 0° C. to about room temperature, to yield the corresponding compound of formula (Va).

Compounds of formula (I) wherein R³ and/or R⁴ are other than hydrogen or R³ and R⁴ are taken together with the nitrogen to which they are bound to form a ring structure, may alternatively be prepared according to the process outlined in Scheme 8.

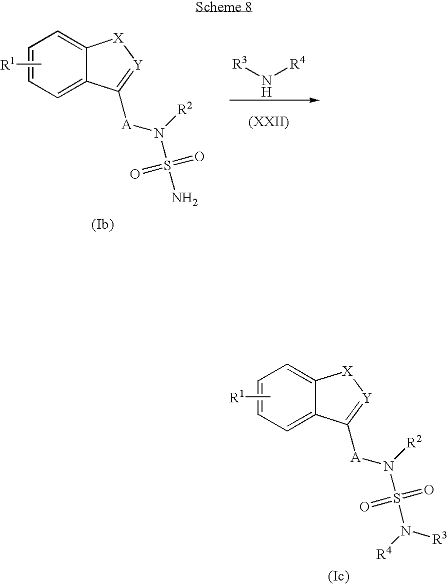

Accordingly, a suitably substituted compound of formula (Ib), is reacted with a suitably substituted amine, a compound of formula (XXII), a known compound or compound prepared by known methods, in water or an organic solvent such as dioxane, ethanol, THF, isopropanol, and the like, provide that the compound of formula (Ib) and the compound of formula (XXII) are at least partially soluble in the water or organic solvent, at a temperature in the range of from about room temperature to about reflux, preferably at about reflux temperature, to yield the corresponding compound of formula (Ic).

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The present invention provides methods of treating epilepsy and related disorders, regardless of underlying cause and stage of development, comprising administering to a subject in need thereof, co-therapy with a therapeutically effective amount of a one or more anticonvulsant or anti-epileptic agents and a therapeutically effective amount of a compound of formula (I) as described herein. The methods of this invention therefore provide the ability to suppress seizures, convulsions or the symptoms of an analogous seizure related disorder. In order to accomplish this objective the compounds or compositions of this invention must be used in the correct therapeutically effective amount or dose, as described below.

Optimal dosages and schedules to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

The present invention further comprises pharmaceutical compositions containing one or more compounds of formula (I) and one or more anti-convulsant and/or anti-epileptic agents with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more of the compounds of formula (I) and more or more of the anticonvulsant and/or anti-epileptic agents, as the active ingredients are intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.1-1000 mg and may be given at a dosage of from about 0.01-200.0 mg/kg/day, preferably from about 0.1 to 100 mg/kg/day, more preferably from about 0.5-50 mg/kg/day, more preferably from about 1.0-25.0 mg/kg/day or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.01 to about 1000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods of the present invention may also be carried out using a pharmaceutical composition comprising any of the compounds as defined herein and a pharmaceutically acceptable carrier. The pharmaceutical composition may contain between about 0.1 mg and 1000 mg, preferably about 50 to 500 mg, of the active compound(s), and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

Compounds of this invention may be administered in any of the foregoing compositions and according to dosage regimens established in the art whenever treatment of epilepsy or related disorders is required.

The daily dosage of the products may be varied over a wide range from 0.01 to 200 mg/kg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 150 mg/kg of body weight per day or any range therein. Preferably, the range is from about 0.1 to about 100 mg/kg of body weight per day, more preferably, from about 0.5 mg/kg to about 50 mg/kg, more preferably, from about 1.0 to about 25.0 mg/kg of body weight per day. The compounds may be administered on a regimen of 1 to 4 times per day.

Therapeutically effective dosage levels and dosage regimens for the anti-convulsant and anti-epileptic agents disclosed herein, may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference (Medical Economics Company or online at http://www.pdrel.com) and other sources.

One skilled in the art will recognize that a therapeutically effective dosage of the compounds of the present invention can include repeated doses within a prolonged treatment regimen that will yield clinically significant results.

One skilled in the art will recognize that, both in vivo and in vitro trials using suitable, known and generally accepted cell and/or animal models are predictive of the ability of a test compound to treat or prevent a given disorder. One skilled in the art will further recognize that human clinical trails including first-in-human, dose ranging and efficacy trials, in healthy patients and/or those suffering from a given disorder, may be completed according to methods well known in the clinical and medical arts.

Determination of effective dosages is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

The following Examples are set forth to aid in the understanding of the invention, and are not intended and should not be construed to limit in any way the invention set forth in the claims which follow thereafter.

### EXAMPLE 1

### N-(benzo[b]thien-3-ylmethyl)-sulfamide (Compound #1)

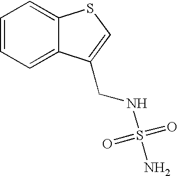

Thianaphthene-3-carboxaldehyde (1.62 g, 10.0 mmol) was dissolved in anhydrous ethanol (50 mL). Sulfamide (4.0 g, 42 mmol) was added and the mixture was heated to reflux for 16 hours. The mixture was cooled to room temperature. Sodium borohydride (0.416 g, 11.0 mmol) was added and the mixture was stirred at room temperature for three hours. The reaction was diluted with water (50 mL) and extracted with chloroform (3×75 mL). The extracts were concentrated and chromatographed (5% methanol in DCM) to yield the title compound as a white solid.

¹H NMR (DMSO-d₆): δ 7.98 (1H, dd, J=6.5, 2.3 Hz), 7.92 (1H, dd, J=6.6, 2.4 Hz), 7.62 (1H, s), 7.36-7.45 (2H, m), 7.08 (1H, t, J=6.3 Hz), 6.72 (2H, s), 4.31 (2H, d, J=6.3 Hz).

### EXAMPLE 2

### N-[(5-chlorobenzo[b]thien-3-yl)methyl]-sulfamide (Compound #3)

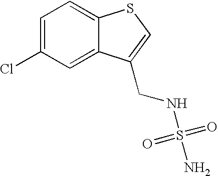

(5-Chloro-1-benzothiophene-3-yl)methylamine (0.820 g, 4.15 mmol) and sulfamide (2.5 g, 26 mmol) were combined in anhydrous dioxane (50 mL) and the mixture was heated to reflux for four hours. The reaction was cooled and diluted with water (50 mL). The solution was extracted with chloroform (3×75 mL). The extracts were concentrated and chromatographed (5% methanol in DCM) to yield the title compound as a white solid.

¹H NMR (DMSO-d₆): δ 8.05 (2H, m), 7.74 (1H, s), 7.40 (1H, d, J=6.5 Hz), 7.07 (1H, t, J=6.3 Hz), 6.72 (2H, s), 4.26 (2H, d, J=6.4 Hz).

### EXAMPLE 3

### N-[(1-methyl-1H-indol-3-yl)methyl]-sulfamide (Compound #7)

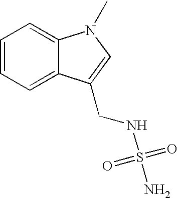

N-Methylindole-3-carboxaldehyde (1.66 g, 10.4 mmol) was dissolved in anhydrous ethanol (50 mL). Sulfamide (4.5 g, 47 mmol) was added and the mixture was heated to reflux for 16 hours. Additional sulfamide (1.0 g, 10.4 mmol) was added and the mixture was heated to reflux for 24 hours. The mixture was cooled to room temperature. Sodium borohydride (0.722 g, 12.5 mmol) was added and the mixture was stirred at room temperature for one hour. The reaction was diluted with water (50 mL) and extracted with DCM (3×75 mL). The extracts were concentrated and about 1 mL of methanol was added to create a slurry which was filtered to yield the title compound as a white powder.

¹H NMR (CD₃OD): δ 7.67 (1H, d, J=5.9 Hz), 7.32 (1H, d, J=6.2 Hz), 7.14-7.19 (2H, m), 7.06 (1H, dt, J=7.7, 0.7 Hz), 4.36 (2H, s), 3.75 (3H, s) MS (M−H)⁻ 237.6.

### EXAMPLE 4

### N-(3-benzofuranylmethyl)-sulfamide (Compound #6)

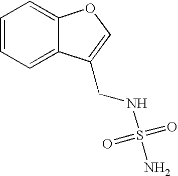

Benzofuran-3-carboxylic acid (1.91 g, 11.8 mmol) was suspended in anhydrous DCM (75 mL). Oxalyl chloride (2.0 M in DCM, 6.48 mL) and then one drop of dimethylformamide were added. The solution was stirred at room temperature for two hours, then ammonium hydroxide (concentrated, 10 mL) was added. The resulting mixture was diluted with water (100 mL) and extracted with DCM (3×100 mL). The extracts were concentrated to a gray solid and dissolved in anhydrous THF (100 mL). Lithium aluminum hydride (1.0 M in THF, 11.8 mL) was added. The mixture was stirred at room temperature for 16 hours. A minimal amount of saturated aqueous NaHCO₃ and then MgSO₄ were added. The mixture was filtered and then extracted with 1 N HCl. The aqueous extracts were adjusted to pH 14 with 3N NaOH and extracted with DCM. The organic extracts were dried with magnesium sulfate and concentrated to a colorless oil. The oil was dissolved in dioxane (50 mL) and sulfamide (3.7 g, 38 mmol) was added. The mixture was heated to reflux for 4 hours, cooled to room temperature, and concentrated. The resulting solid was chromatographed (5% methanol in DCM) to yield the title compound as a slightly yellow solid.

¹H NMR (CD₃OD): δ 7.53 (1H, d, J=5.7 Hz), 7.44 (1H, d, J=6.0 Hz), 7.16-7.26 (2H, m), 6.73 (1H, s), 4.35 (2H, s).

### EXAMPLE 5

### N-[(5-fluorobenzo[b]thien-3-yl)methyl]-sulfamide (Compound #8)

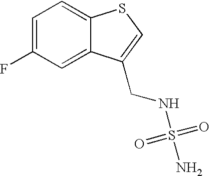

5-Fluoro-3-methylbenzothiophene (1.14 g, 6.83 mmol), benzoyl peroxide (0.165 g, 0.68 mmol) and N-bromosuccinimide (1.70 g, 7.52 mmol) were combined in carbon tetrachloride (25 mL) and the mixture was heated to reflux for 3 hours. The yellow solution was cooled, diluted with water, and extracted with DCM (2×50 mL). The extracts were washed with brine (100 mL), dried with magnesium sulfate, and concentrated to an orange solid. The solid was dissolved in anhydrous DMF. Sodium azide (4.0 g, 61 mmol) was added and the mixture was stirred for 16 hours at room temperature. The reaction was diluted with water (100 mL) and extracted with diethyl ether (2×75 mL). The extracts were washed with brine (100 mL), dried with magnesium sulfate, and concentrated to a yellow oil. The oil was dissolved in a mixture of THF (50 mL) and water (5 mL). Triphenylphosphine (3.60 g, 13.7 mmol) was added. The mixture was stirred at room temperature for 16 hours. The reaction was concentrated and chromatographed (2 to 5% methanol in DCM). The resulting C-(5-fluoro-benzo[b]thien-3-yl)-methylamine (1.04 g, 5.73 mmol) was dissolved in anhydrous dioxane (50 mL) and sulfamide (2.75 g, 28.7 mmol) was added. The reaction was heated to reflux for 4 hours, cooled to room temperature, and concentrated to a solid which was chromatographed (5% methanol in DCM) to yield the title compound as a white solid.

¹H NMR (CD₃OD): δ 7.85 (1H, dd, J=6.6, 3.6 Hz), 7.66 (1H, dd, J=7.4, 1.8 Hz), 7.62 (1H, s), 7.13-7.18 (1H, m), 4.40 (2H, s).

### EXAMPLE 6

### N-(1-benzo[b]thien-3-ylethyl)-sulfamide (Compound #9)

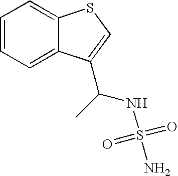

3-Acetylthianaphthene (3.00 g, 17.0 mmol) was added to a mixture of formic acid (10 mL) and formamide (10 mL). The solution was heated to 150° C. for 8 hours. The reaction was cooled to room temperature, diluted with water (50 mL), and extracted with diethyl ether (3×50 mL). The ether extracts were washed with saturated aqueous NaHCO₃ and brine. The solution was concentrated and chromatographed (5% methanol in DCM) to yield N-(1-benzo[b]thiophen-3-yl-ethyl)-formamide (1.76 g) as a white solid which was suspended in concentrated HCl (30 mL). The mixture was heated to reflux for 1.5 hours then diluted with water (100 mL). 3N NaOH was added until the pH was 14. The mixture was extracted with diethyl ether (3×100 mL) then dried with magnesium sulfate and concentrated to an orange oil. The oil was dissolved in anhydrous dioxane (75 mL) and sulfamide was added. The mixture was heated to reflux for 2 hours then diluted with water (50 ml). The solution was extracted with ethyl acetate (2×50 mL), dried with magnesium sulfate, concentrated, and chromatographed (2.5% to 5% methanol in DCM) to yield the title compound as a white solid.

¹H NMR (CD₃OD): δ 8.01 (1H, dd, J=5.5, 0.7 Hz), 7.85 (1H, dt, J=6.0, 0.6 Hz), 7.49 (1H, s), 7.31-7.40 (2H, m), 4.95 (1H, q, J=5.1 Hz), 1.67 (3H, d, J=5.1 Hz).

### EXAMPLE 7

### N-(1-naphthalenylmethyl)-sulfamide (Compound #10)

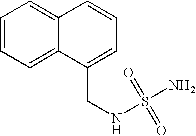

1-Naphthanlenemethylamine (2.00 g, 12.7 mmol) and sulfamide (5.0 g, 52 mmol) were combined in anhydrous dioxane (100 mL) and the mixture was heated to reflux for 6 hours. The reaction was cooled to room temperature and was filtered. The filtrate was concentrated to a solid and washed with water until TLC indicated no remaining trace of sulfamide in the solid. The collected solid was dried under vacuum to yield the title compound as a white solid.

¹H NMR (CDCl₃): δ 8.09 (1H, d, J=6.3 Hz), 7.86 (1H, dd, J=12.9, 6.2 Hz), 7.42-7.61 (4H, m), 4.75 (2H, d, J=4.4 Hz), 4.58 (1H, br s), 4.51 (2H, br s).

### EXAMPLE 8

### N-[(2-methyl-3-benzofuranyl)methyl]-sulfamide (Compound #13)

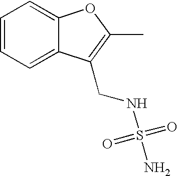

2-Methylbenzofuran-3-carbaldehyde (0.51 g, 3.18 mmol) was dissolved in anhydrous ethanol (25 mL). Sulfamide (1.5 g, 16 mmol) was added and the mixture was heated to reflux for 4 days. The mixture was cooled to room temperature. Sodium borohydride (0.132 g, 3.50 mmol) was added and the mixture was stirred at room temperature for 24 hours. The reaction was diluted with water (100 mL) and extracted with DCM (3×75 mL). The extracts were concentrated and suspended in a minimal amount of DCM and filtered to yield the title compound as a white solid.

¹H NMR (DMSO-d₆): δ 7.65 (1H, dd, J=6.4, 2.6 Hz), 7.43-7.47 (1H, m), 7.19-7.23 (2H, m), 6.87 (1H, t, J=6.2Hz), 6.68 (2H, s), 4.11 (2H, d, J=6.2 Hz), 2.42 (3H, s).

### EXAMPLE 9

### N-[(5-bromobenzo[b]thien-3-yl)methyl]-sulfamide (Compound #15)

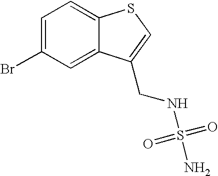

5-Bromobenzothiophene (1.60 g, 7.51 mmol) and dichloromethyl methyl ether (1.29 g, 11.3 mmol) were dissolved in anhydrous 1,2-dichloroethane (75 mL). Titanium tetrachloride (2.14 g, 11.3 mmol) was added, turning the solution dark. After one hour at room temperature, the reaction was poured into a mixture of saturated aqueous NaHCO₃ and ice. The mixture was stirred for about 30 minutes and then was extracted with DCM (2×100 mL). The extracts were concentrated and chromatographed (0 to 5% ethyl acetate in hexane) to yield 5-bromo-benzo[b]thiophene-3-carbaldehyde (1.32 g). The 5-bromobenzothiophene-3-carboxaldehyde (1.20 g, 4.98 mmol) and sulfamide (4.0 g, 42 mmol) were combined in anhydrous ethanol (25 mL) and heated to reflux for three days. The reaction was cooled to room temperature and sodium borohydride (0.207 g, 5.47 mmol) was added. After five hours, water (50 ml) was added and the solution was extracted with chloroform (3×50 mL). The extracts were concentrated, suspended in a minimal amount of DCM, and filtered to provide the title compound as a yellow solid.

¹H NMR (DMSO-d₆): δ 8.12 (1H, d, J=1.8 Hz), 7.97 (1H, d, J=8.6), 7.71 (1H, s), 7.52 (1H, dd, J=8.6, 1.9 Hz), 7.12 (1H, t, J=6.3 Hz), 6.72 (2H, s), 4.28 (2H, d, J=6.2 Hz).

### EXAMPLE 10

### N-[(4-bromobenzo[b]thien-3-yl)methyl]-sulfamide (Compound #17)

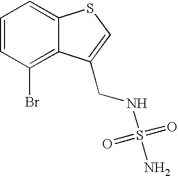

4-Bromobenzothiophene (1.8 0 g, 8.45 mmol) and dichloromethyl methyl ether (1.46 g, 12.7 mmol) were dissolved in anhydrous DCM (100 mL). Titanium tetrachloride (2.40 g, 12.7 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×150 mL). The extracts were concentrated and chromatographed (0 to 15% ethyl acetate in hexane) to yield 4-bromobenzothiophene-3-carboxaldehyde (0.910 g). The 4-bromobenzothiophene-3-carboxaldehyde (0.910 g, 3.77 mmol) and sulfamide (3.0 g, 31 mmol) were combined in anhydrous ethanol (25 mL) and heated to reflux for three days. The reaction was cooled to room temperature and sodium borohydride (0.157 g, 4.15 mmol) was added. After five hours, water (50 ml) was added and the solution was extracted with chloroform (3×50 mL). The extracts were concentrated, suspended in a minimal amount of DCM, and filtered to yield the title compound as a yellow solid.

¹H NMR (DMSO-d₆): δ 8.05 (1H, dd, J=8.1, 0.8 Hz), 7.78 (1H, s), 7.64 (1H, dd, J=7.6, 0.8 Hz), 7.27 (1H, t, J=7.9 Hz), 7.13 (1H, t, J=6.3 Hz), 6.72 (2H, br s), 4.65 (2H, d, J=5.3 Hz).

### EXAMPLE 11

### N-[(7-fluorobenzo[b]thien-3-yl)methyl]-sulfamide (Compound #18)

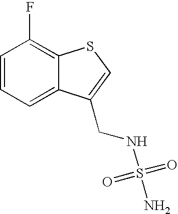

2-Fluorothiophenol (4.14 g, 32.6 mmol) was dissolved in anhydrous THF (100 mL). Potassium tert-butoxide (1.0 M in THF, 35.8 mL) was added and the suspension was stirred at room temperature for 15 minutes. 2-Chloroacetaldehyde dimethyl acetal was added and the mixture was stirred for 3 days. Water (100 mL) was added and the solution was extracted with diethyl ether (3×100 mL). The extracts were concentrated to a yellow oil and chromatographed (5 to 20% ethyl acetate in hexane) to yield 1-(2,2-dimethoxy-ethylsulfanyl)-2-fluoro-benzene (6.42 g) as a colorless oil. Chlorobenzene (25 mL) was heated to reflux and polyphosphoric acid (1 mL) was added. The 1-(2,2-dimethoxy-ethylsulfanyl)-2-fluoro-benzene was then added slowly turning the solution dark. After 3 hours of heating, the reaction was cooled to room temperature and diluted with water (50 mL). The solution was extracted with benzene (2×50 mL). The extracts were concentrated and chromatographed (0 to 15% ethyl acetate in hexane) to yield 7-fluorobenzothiophene (0.77 g). The 7-fluorobenzothiophene (0.77 g, 5.1 mmol) and dichloromethyl methyl ether (0.872 g, 7.6 mmol) were dissolved in anhydrous DCM (25 mL). Titanium tetrachloride (1.0 M in DCM, 7.6 mL, 7.6 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×50 mL). The extracts were concentrated and chromatographed (0 to 15% ethyl acetate in hexane) to yield 7-fluorobenzothiophene-3-carboxaldehyde (0.642 g). The 7-fluorobenzothiophene-3-carboxaldehyde (0.642 g, 3.77 mmol) and sulfamide (1.7 g, 18 mmol) were combined in anhydrous ethanol (20 mL) and heated to reflux for three days. The reaction was cooled to room temperature and sodium borohydride (0.148 g, 3.92 mmol) was added. After two hours, water (25 ml) was added and the solution was extracted with chloroform (3×25 mL). The extracts were concentrated, suspended in a minimal amount of DCM, and filtered to yield the title compound as a yellow solid.

¹H NMR (DMSO-d₆): δ 7.78 (1H, d, J=8.0 Hz), 7.43-7.50 (1H, m), 7.27 (1H, dd, J=10.3, 7.9 Hz), 7.14 (1H, t, J=6.4 Hz), 6.74 (2H, brs), 4.31 (2H, d, J=6.4 Hz).

### EXAMPLE 12

### N-[(4-trifluoromethylbenzo[b]thien-3-yl)methyl]-sulfamide (Compound #19)

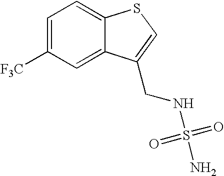

4-Trifluoromethylbenzothiophene (0.276 g, 1.37 mmol) and dichloromethyl methyl ether (0.236 g, 2.06 mmol) were dissolved in anhydrous DCM (10 mL). Titanium tetrachloride (1.0 M in DCM, 2.1 mL, 2.1 mmol) was added, turning the solution dark. After 30 minutes at room temperature, the reaction was poured into a mixture of saturated aqueous NaHCO₃ and ice. The mixture was stirred for about 30 minutes and then extracted with DCM (2×25 mL). The extracts were concentrated and chromatographed (0 to 15% ethyl acetate in hexane) to yield 4-trifluoromethylbenzothiophene-3-carboxaldehyde.

The 4-trifluoromethylbenzothiophene-3-carboxaldehyde (0.226 g, 0.982 mmol) and sulfamide (0.471 g, 4.91 mmol) were combined in anhydrous ethanol (5 mL) and heated to reflux for 24 hours. The reaction was cooled to room temperature and sodium borohydride (0.056 g, 1.47 mmol) was added. After five hours, water (10 ml) was added and the solution was extracted with chloroform (3×10 mL). The extracts were concentrated, and chromatographed (5% methanol in DCM) to yield the title compound as a white solid.

¹H NMR (DMSO-d₆): δ 8.30 (1H, s), 8.25 (1H, d, J=8.4 Hz), 7.84 (1H, s), 7.68 (1H, dd, J=8.5, 1.4 Hz), 6.7-6.9 (2H, br s), 4.4-4.5 (1H, br s), 4.37 (2H, s).

### EXAMPLE 13

### N-[(4-cyanobenzo[b]thien-3-yl)methyl]-sulfamide (Compound #20)

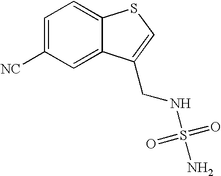

4-Cyanobenzothiophene (1.15 g, 7.22 mmol) and dichloromethyl methyl ether (1.25 g, 10.8 mmol) were dissolved in anhydrous DCM (100 mL). Titanium tetrachloride (1.0 M in DCM, 10.8 mL, 10.8 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×50 mL). The extracts were concentrated and chromatographed (O to 15% ethyl acetate in hexane) to yield 4-cyanobenzothiophene-3-carboxaldehyde.

The 4-cyanobenzothiophene-3-carboxaldehyde (0.298 g, 1.59 mmol) and sulfamide (0.766 g, 7.97 mmol) were combined in anhydrous ethanol (20 mL) and heated to reflux for 24 hours. The reaction was cooled to room temperature and sodium borohydride (0.091 g, 2.39 mmol) was added. After five hours, water (20 ml) was added and the solution was extracted with chloroform (3×20 mL). The extracts were concentrated, and chromatographed (5% methanol in DCM) to yield the title compound as a white solid.

¹H NMR (DMSO-d₆): δ 8.37 (1H, s), 8.30 (1H, d, J=8.4 Hz), 7.87 (1H, s), 7.70 (1H, dd, J=8.5, 1.4 Hz), 6.7-6.9 (2H, br s), 4.4-4.5 (1H, br s), 4.40 (2H, s).

### EXAMPLE 14

### N-[(benzo[b]thien-3-yl)methyl]-sulfamoylpyrrolidine (Compound #101)

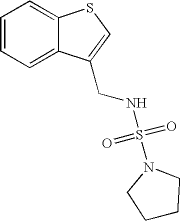

N-[(Benzo[b]thien-3-yl)methyl]-sulfamide (0.250 g, 1.03 mmol) and pyrrolidine (0.25 mL) were combined in anhydrous dioxane (5 mL) and heated to reflux for 32 hours. The reaction was evaporated and chromatographed with 5% methanol in DCM to yield the title compound as a white solid.

¹H NMR (CDCl₃): δ 7.84-7.89 (2H, m), 7.38-7.45 (3H, m), 4.49 (3H, br s), 3.25 (4H, t, J=4.0 Hz), 1.80 (4H, t, J=4.0 Hz).

### EXAMPLE 15

### N-[(benzo[b]thien-3-yl)methyl]-N′-ethylsulfamide (Compound #21)

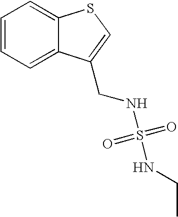

N-[(Benzo[b]thien-3-yl)methyl]-sulfamide (0.250 g, 1.03 mmol) and ethylamine (70% in H₂O, 0.10 mL) were combined in anhydrous dioxane (5 mL) and heated to reflux for 32 hours. The reaction was evaporated and chromatographed with 5% methanol in DCM to yield the title compound as a white solid.

¹H NMR (CDCl₃): δ 7.83-7.90 (2H, m), 7.36-7.47 (3H, m), 4.51 (2H, s), 2.90 (2H, q, J=7 Hz), 1.03 (3H, t, J=7 Hz).

### EXAMPLE 16

### Imidazole-1-sulfonic acid [(benzo[b]thien-3-yl)methyl]-amide (Compound #102)

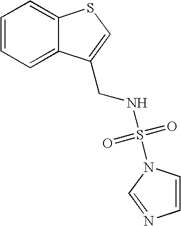

3-Benzothienylmethylamine and 3-(imidzole-1-sulfonyl)-1-methyl-3H-imidazol-1-ium triflate were combined in anhydrous acetonitrile. The solution was stirred at room temperature overnight, concentrated, and chromatographed (5% methanol in DCM) to yield the title compound as a tan solid.

¹H NMR (DMSO-d₆): δ 8.05 (1H, dd, J=7.0, 1.6 Hz), 7.99 (1H, dd, J=7.1, 1.7 Hz), 7.85 (1H, s), 7.66 (1H, s), 7.42-7.65 (5H, m), 4.34 (2H, s).

### EXAMPLE 17

### Prophetic Example Non Randomized, Within Subject Placebo Controlled Study: Photo-Induced Paroxysnal EEG Response in Patients with Photosensitive Epilepsy

Rationale for Study:

Photosensitivity offers a useful model for acute antiepileptic drug studies in man. The technique of using the photosensitive range as an index for antiepileptic action has been proven to be effective with a number of well-known antiepileptic drugs. In addition, it appears to be a useful tool for preliminary investigation of new potential antiepileptic drugs (Binnie et al., 1985; Kasteleijn-Nolst Trenité et al., 1996). Apart from information concerning the efficacy of the antiepileptic drug, the technique may, when combined with continuous blood level monitoring, also offer information concerning the time of onset and the duration of the antiepileptic action. In some cases the maximal reduction of the photosensitive range is not concurrent with, but delayed in relation to the time of the peak blood levels of a drug, as for example in the case of sodium valproate.

Using the classical photoparoxysmal response (generalised spikes, spikewaves, or polyspikewaves), as a model, the effect of the experimental antiepileptic drug on the distribution of epileptiform activity may help predict the clinical anti-convulsive spectrum of the new drug. It may lead to complete abolishment of the photoparoxysmal response, or alternatively, it may also result in the inhibition of the secondary spread and generalisation of the primary epileptiform discharges in the occipital lobe (Binnie et al., 1986).

Objective:

The objectives of the Study are as follows:

(a) to evaluate the acute antiepileptic effects of test compound (i.e. a compound of formula (I)) in photosensitive epilepsy patients, using the photoparoxysmal EEG response to intermittent photic stimulation (IPS) as a marker of antiepileptic activity; (b) to determine an oral dose of test compound (i.e. a compound of formula (I)) that results in complete suppression of photosensitivity, or reduces the photosensitivity range by at least 3 points on the photosensitivity scale in at least one eye condition (open, closure, closed); (c) to assess the relationship of the antiepileptic effect to plasma levels of test compound (i.e. a compound of formula (I)); (d) to investigate possible interactions with pre-existing antiepileptic drugs (AED); (e) to provide information on the safety and tolerability of the test compound (i.e. a compound of formula (I)) in patients with photosensitive epilepsy; and (f) to investigate the acute effect of the test compound (i.e. a compound of formula (I)) on mood in patients with photosensitive epilepsy.

Overview of the Study:

The study is a multi-center, non-randomized, single-blind, within subject placebo controlled study. All subjects receive a single dose of placebo on the morning of Day 1, a single dose of test compound (i.e. a compound of formula (I)) on the morning of Day 2 and a second single dose of placebo on the morning of Day 3. EEG tracings, recorded during IPS sessions, are printed on paper, coded and evaluated independently by 2 blinded investigators to determine the effects on the photosensitivity range.

The dose of the test compound (i.e. a compound of formula (I)) in the first three patients is selected based on animal studies. If there is a complete suppression of photosensitivity or reduction of the photosensitivity range by at least 3 points on the photosensitivity scale in at least 2 of these 3 subjects, the dose of test compound (i.e. a compound of formula (I)) is reduced in the next 3 subjects. The dose of the test compound (i.e. a compound of formula (I)) is reduced in stepwise fashion (down to a minimum dose of 250 mg) until reduction or suppression of photosensitivity is not seen, or is seen in fewer than 2 out of 3 subjects in the last dose level tested.

Once the steps above are completed, if complete suppression of photosensitivity is not seen in 2 out of the first 3 subjects at the initial dose level, the dose of the test compound (i.e. a compound of formula (I)) is increased in the next 3 subjects. The dose of the test compound (i.e. a compound of formula (I)) is increased in stepwise fashion until complete suppression of photosensitivity is seen in at least 2 subjects. These dose increases are performed only if the previous dose level is well tolerated and the new dose level is supported by safety and tolerance data from the healthy volunteer studies. In addition, these dose increases are performed only once plasma levels of the test compound (i.e. a compound of formula (I)) are known and have been compared with data from the healthy volunteer studies.

Study Population:

Up to 18 male or female subjects (3 per dose level), between 16 and 60 years of age, and with a firm diagnosis of idiopathic, photosensitive epilepsy (as characterized by a diffuse photoparoxysmal EEG response), which is not associated with mental defects or brain lesions. Subjects not using antiepileptic medication will be preferred, but use of antiepileptic medication (with the exception of felbamate) is not an exclusion criterion.

For participations in the study, each subject must satisfy the following criteria, before entering the study:
(a) aged 16 to 60 years inclusive
(b) has read and signed the informed consent form
(c) body weight between 40 and 90 kg (inclusive)
(d) firm documented diagnosis of idiopathic, photosensitive epilepsy with a diffuse photoparoxysmal EEG response
(e) consistent sensitivity to intermittent photic stimulation over a suitable range of flash frequencies
(f) no relevant abnormal clinical laboratory tests
(g) likely to be able to take part in the whole study.

Subjects who meet any of the following criteria are excluded from participating in the study:
(a) known chronic infections or allergies or history of severe allergy
(b) pregnant or lactating female or female insufficiently protected against pregnancy (for female subjects of childbearing potential, a negative pregnancy test must be obtained and either abstinence or two reliable methods of contraception must be used starting from at least 2 weeks prior to study drug administration and continuing until at least 1 week after study completion)
(c) any serious illness other than epilepsy
(d) significant neurological, psychiatric or learning disability
evidence of progressive brain lesion (eg. on brain MRI or CT if appropriate)
systolic blood pressure >160 or <90 mmHg and diastolic blood pressure >95 or <60 mmHg according to two repeated measures within 10 min interval
regular use of non-topical medications other than current antiepileptic drugs (except felbamate) or oral contraceptives within 7 days prior to study drug administration (non-prescription OTC treatments can be accepted according to the investigator's judgement)
participation in a clinical trial or use of an experimental drug within 60 days prior to study drug administration
(i) use of neuroleptics (typical or atypical), anti-depressants or Felbamate within 60 days prior to study drug administration
(j) use of more than two antiepileptic medications or change in antiepileptic medication within 30 days prior to study drug administration
(k) acute use of an antiepileptic medication within 7 days prior to study drug administration
(l) history of alcohol abuse or drug addiction within 90 days prior to study drug administration
(m) legal incapacity or limited legal capacity
(n) likely not to cooperate with or to respect the constraints of the study.

Subjects taking concomitant antiepileptic medications (with the exception of felbamate) and who are stable will continue with their regular medications maintained at the same dose level. All concomitant medication (prescription and non-prescription) will be recorded.

Dosage and Administration:

Subjects are dosed orally at approximately 09:00 hrs each day, with a standard glass of water (240 ml), under the supervision of the investigator or designated study personnel. The exact time of administration and correct intake of the capsules will be noted and recorded in the CRF.

Before any study specific procedures is conducted, the subjects reads and signs a Written Informed Consent Form. During the screening period, within 30 days prior to study drug administration, the following assessments are completed for each subject:
(a) Medical history (including seizure history).
(b) Physical examination (including neurological examination, vital signs: standing and supine blood pressure, heart rate, weight, height and oral temperature).
(c) All medications (prescription and non-prescription), including antiepileptic drugs, used within 30 days prior to study drug administration.
(d) Full diagnostic routine EEG work-up, including control EEG and standardized determination of the photosensitivity range.
(e) 12-lead ECG.
(f) for subjects receiving concomitant antiepileptic drugs (AEDs), a blood sample for analysis of AED levels will be taken.

Standard clinical laboratory assessments include:
(a) hematology: haemoglobin, hematocrit, erythrocytes, mean corpuscular volume (MCV), mean corpuscular haemoglobin mass (MCH), mean corpuscular haemoglobin concentration (MCHC), leukocytes (total WBC and automated differential counts), platelet count.
(b) clinical chemistry: gamma glutamyl transpeptidase (yGT), alanine aminotransferase (ALT), aspartate aminotransferase (AST), alkaline phosphatase, LDH, creatinine, uric acid, glucose, total bilirubin, total protein, albumin, cholesterol, triglycerides, urea, sodium, potassium, calcium, chloride.
(c) urinalysis: glucose, protein, blood, bicarbonate, citrate, pH. If abnormal protein or blood values are found, a microscopic inspection will be performed.
   Single-Blind Treatment Phase:

Subjects who have completed the screening assessments and who meet the inclusion/exclusion criteria are admitted to the hospital for the treatment phase. The duration of this treatment phase is 3 consecutive days, during which subjects are confined to the clinic for observation. Unscheduled EEG monitoring may be performed during this period at the investigator's discretion. All adverse events (AE), including seizures, are recorded between the time of first admission on Day 1 and the end of study on Day 3 (see Section 10).

On each of the three treatment days subjects are instructed to eat breakfast no later than 7:00 am (two hours before study drug administration). The breakfast should consist of a light meal (ie., dry cereal, juice, coffee/tea); fatty foods should be avoided (ie., cheese, pork, large amounts of butter/margarine, whole milk or cream). Lunch is provided at approximately 12:00, noon, and contains a balanced combination of food groups. Foods that may precipitate a hypersensitive reaction with neurological complications are avoided (ie., ergot amine containing cheeses).

Day 1: Subjects are admitted to the hospital by approximately 08:00 hrs. EEG electrodes will be put in place. For female subjects of childbearing potential a urine sample is obtained and pregnancy test performed prior to dose administration. Standard clinical laboratory assessments (as described for the screening phase) are performed within 1 hour prior to study drug administration. A single oral dose of placebo is administered at approximately 09:00 hrs. To determine the photosensitivity range, IPS and 21-channel EEG recordings are performed shortly before study drug administration and at hourly intervals up to 8 hours post-dose, following a standardized procedure. For subjects receiving concomitant antiepileptic drugs (AEDs), blood samples for analysis of AED levels are taken immediately before administration of study drug and at hourly intervals (immediately following each IPS assessment) up to 8 hours post-dose. Vital signs (standing and supine blood pressure, pulse) are recorded within 1 hour prior to study drug administration and at 1, 3, 6 and 8 hours post-dose (after photosensitivity assessments and pharmacokinetic blood sampling have been performed). A standard neurological examination is performed at 4 hours post-dose (after photosensitivity assessments and pharmacokinetic blood sampling have been performed). The POMS questionnaire wisbe administered within 1 hour prior to study drug administration and at 1, 3 and 6 hours post-dose (after photosensitivity assessments and pharmacokinetic blood sampling have been performed).

Day 2: Immediately before dosing on Day 2 subjects are instructed to void their bladders. This urine is discarded and the 10-hour urine collection period begins. All urine is collected until 10-hours post-dose. A single oral dose of test compound (i.e. a compound of formula (I)) is administered at approximately 09:00 hrs. To determine the photosensitivity range, IPS and 21-channel EEG recordings are performed shortly before study drug administration and at hourly intervals up to 8 hours post-dose, following a standardized procedure. Blood samples for analysis of test compound (i.e. a compound of formula (I)) levels are taken immediately before administration of study drug and at hourly intervals (immediately following each IPS assessment) up to 8 hours post-dose. For subjects receiving concomitant antiepileptic drugs (AEDs), blood samples for analysis of AED levels are taken immediately before administration of study drug and at hourly intervals (immediately following each IPS assessment) up to 8 hours post-dose. Vital signs (standing and supine blood pressure, pulse) are recorded within 1 hour prior to study drug administration and at 1, 3, 6 and 8 hours post-dose (after photosensitivity assessments and pharmacokinetic blood sampling have been performed). A standard neurological examination is performed at 4 hours post-dose (after photosensitivity assessments and pharmacokinetic blood sampling have been performed). The POMS questionnaire is administered within 1 hour prior to study drug administration and at 1, 3 and 6 hours post-dose (after photosensitivity assessments and pharmacokinetic blood sampling have been performed). At 10 hours post-dose the subjects are instructed to void their bladders to complete the 10-hour urine collection. The total volume of urine collected is measured and an aliquot removed for exploratory metabolite analysis.

Day 3: A single oral dose of placebo is administered at approximately 09:00 hrs. To determine the photosensitivity range, IPS and 21-channel EEG recordings is performed shortly before study drug administration and at hourly intervals up to 8 hours post-dose, following a standardized procedure. In order to examine duration of effects of test compound (i.e. a compound of formula (I)) given on Day 2, blood samples for analysis of test compound (i.e. a compound of formula (I)) levels are taken immediately before administration of the placebo dose on Day 3 and at hourly intervals (immediately following each IPS assessment) up to 8 hours post-dose on Day 3. For subjects receiving concomitant antiepileptic drugs (AEDs), blood samples for analysis of AED levels are taken immediately before administration of study drug and at hourly intervals (immediately following each IPS assessment) up to 8 hours post-dose. Vital signs (standing and supine blood pressure, pulse) are recorded within 1 hour prior to study drug administration and at 1, 3, 6 and 8 hours post-dose (after photosensitivity assessments and pharmacokinetic blood sampling have been performed). A standard neurological examination is performed at 4 hours post-dose (after photosensitivity assessments and pharmacokinetic blood sampling have been performed). The POMS questionnaire is administered within 1 hour prior to study drug administration and at 1, 3 and 6 hours post-dose (after photosensitivity assessments and pharmacokinetic blood sampling have been performed). A physical examination (including oral temperature), 12-lead ECG and standard clinical laboratory assessments (as described for the screening phase) are performed 8 hours post-dose, prior to discharge, after all previous assessments have been completed.

Posttreatment Phase (Follow-Up): Any adverse events or clinically significant laboratory abnormalities persisting at the end of the study on Day 3 are followed until resolution, or until reaching a clinically stable endpoint. If the adverse events or laboratory abnormalities can be attributed to factors other than the study drug and other than study conduct, no further follow up will be required.

Pharmocokinetic/Pharmacodynamic Evaluations:

Blood samples for assessment of plasma levels of the test compound (i.e. a compound of formula (I)) are taken immediately before administration of study drug on Day 2 and at hourly intervals (immediately following each IPS assessment) up to 8 hours post-dose. In order to examine duration of effects of the test compound (i.e. a compound of formula (I)) given on Day 2, blood samples for analysis of the test compound (i.e. a compound of formula (I)) levels are taken immediately before administration of the placebo dose on Day 3 and at hourly intervals (immediately following each IPS assessment) up to 8 hours post-dose on Day 3. For subjects receiving concomitant antiepileptic drugs (AEDs), blood samples for analysis of AED levels are taken immediately before administration of study drug and at hourly intervals (immediately following each IPS assessment) up to 8 hours post-dose on Days 1, 2 and 3. The relationship of antiepileptic effect and adverse events to plasma level, and interactions with pre-existing antiepileptic drugs are evaluated.

For determination of test compound (i.e. a compound of formula (I)) and AED concentrations, each 5-10 ml blood sample is drawn from a peripheral vein into sodium heparinized tubes and centrifuged within 15 minutes of collection for at least 15 minutes at approximately 3000 rpm in a refrigerated centrifuge. The plasma is separated into two aliquots (at least 1.2 ml each) and placed in labeled polypropylene tubes. Plasma samples are stored at −20° C. until analysis. Total volumes of the 24-hour urine collections are measured. A 250 ml sample is removed, labeled and frozen for exploratory metabolite analysis. Plasma samples are analyzed to determine concentration of test compound (i.e. a compound of formula (I)) using a validated, specific and sensitive LC-MS/MS method. Analysis of samples for determination of concomitant AED concentrations is completed by standard techniques at a central laboratory.

Plasma concentrations of test compound (i.e. a compound of formula (I)) are determined immediately before administration of study drug on day 2 and at hourly intervals (immediately following each IPS assessment) up to 8 hours post-dose. In order to examine duration of effects of test compound (i.e. a compound of formula (I)) given on Day 2, blood samples for analysis of test compound (i.e. a compound of formula (I)) levels are also taken immediately before administration placebo dose on Day 3 and at hourly intervals (immediately following each IPS assessment) up to 8 hours post-dose on Day 3. For patients receiving concomitant AEDs, AED levels will be determined from samples taken before administration of study drug and at hourly intervals (immediately following each IPS assessment) up to 8 hours post-dose on days 1, 2 and 3.

Intermittent photic stimulation (IPS) are performed to determine the photosensitivity range pre-dose and at hourly intervals up to 8 hours post-dose on days 1, 2 and 3. The IPS assessment follwos a standard procedure using a Grass-type PS 22 photic stimulator with an unpatterned lamp glass at a distance from the nasion of approximately 300 mm and with an intensity of 100 cd/m²/flash. Subjects are seated and instructed to fixate on the center of the lamp. Trains of flashes at constant frequency are delivered for 4-6 seconds. Intervals between the successive flash trains at a given frequency last at least 5 seconds. The following frequencies are tested: 2, 4, 8, 10, 13, 15, 18, 20, 23, 25, 30, 40, 50 and 60 Hz. First the lower limit is established by starting with 2-Hz stimulation and testing successive increasing standard frequencies (as defined above) until epileptiform activity is elicited. Then the upper sensitivity limit is defined, beginning at 60 Hz and decreasing the flash frequency in a stepwise manner until diffuse/generalized epileptiform activity is again elicited. IPS sensitivity is tested for each of three eye conditions: open, during closure, closed. A change in photosensitivity is calculated from the differences in the sensitivity range on the scale of frequencies given above (each frequency tested represents one point on the scale). For example, a change from 10 and 25 Hz (lower and upper limits) to 18 and 20 Hz would give a difference of 3+2=5 points.

As soon as diffuse/generalized EEG epileptiform activity appears, the stimulation at the frequency in question is terminated. This procedure is performed in a hospital setting under the supervision of a qualified physician. It very rarely results in actual seizure activity. If a seizure should occur, trained and experienced medical staff is on hand to intervene as required. If any subject does experience a seizure during the IPS procedure, that subject is withdrawn from the study. IPS sessions are monitored and recorded on video.

Mood is determined using the Profile of Mood States (POMS) instrument. The POMS is a self-administered scale of general psychopathology, consisting of 65 ordinal items (Educational and Industrial Testing Service, San Diego, California). In the POMS the subject checks one of the 5 degrees of each item:
0=Not at all
1=A little
2=Moderately
3=Quite a bit
4=Extremely

During the pre-study visit, the questionnaire is presented to the subject, but not completed. Explanations on the manner to complete the questionnaire are given. It usually suffices to make sure the instructions are clear and then leave the POMS with the subject to complete. The examiner is available in case questions arise. Questions are answered, but the examiner avoids defining one POMS item by referring to any other POMS item. Most subjects complete the POMS in about three-five minutes. At the end, the examiner checks that all items have been answered.

A factorial analysis isolates 6 factors:
Tension-Anxiety: items 2, 10, 16, 20, 22, 26, 27, 34, 41
Depression-Dejection: items 5, 9, 14, 18, 21, 23, 32, 35, 36, 44, 45, 48, 58, 61, 62
Anxiety-Hostility: items 3, 12, 17, 24, 31, 39, 42, 47, 52, 53, 57
Fatigue: items 4, 11, 29, 40, 46, 49, 65
Vigor: items 7, 15, 19, 38, 51, 56, 60, 63
Confusion: items 8, 28, 37, 50, 54, 59, 64

The sum of the items corresponding to each factor is calculated. Safety Evaluations:

Standard clinical laboratory assessments (biochemistry, hematology and urinalysis) are performed at the screening visit, within 1 hour prior to study drug administration on Day 1 and 8 hours after study drug administration (prior to discharge) on Day 3. Vital signs (blood pressure and pulse) are assessed at the screening visit, within 1 hour prior to study drug administration and at 1, 3, 6 and 8 hours post-dose (after photosensitivity assessment and pharmacokinetic blood sampling have been performed) on Days 1, 2 and 3. A standard 12-lead ECG and physical examination, including oral temperature is performed at the screening visit and immediately prior to discharge on Day 3. Standard neurological examination is performed at screening and at 4 hours post-dose on Days 1, 2 and 3. Adverse events are reported between the time of first admission on Day 1 and the end of study on Day 3.

For all the subjects included in the study, demographic data (sex, age, weight height and body mass index) as well as results of P₄₅₀ genotyping and diagnostic EEG are summarised using descriptive statistics. Abnormal medical history will only be listed by subject. For physical examination and standard neurological examination all anomalies are listed. Frequency tables are computed for baseline examinations and for all changes from the baseline examination results.

Clinical laboratory, ECG and vital signs (including oral temperature) are summarised for pre-study (selection phase) and for changes from baseline to each timepoint of assessment. ECG, vital sign and laboratory values out of normal range are flagged in the listings and their frequency summarised if applicable. Shifts of laboratory values from pre-study to end of study in low/normal/high categories are summarised. Vital signs are presented graphically as individual profiles over time.

Clinical safety evaluation is based upon the review of individual values (ECG, vital signs, blood and urine analysis), values outside normal range (ECG, vital signs, blood and urine analysis) and descriptive statistics (summary tables, graphics).

Adverse Events (AEs) are reported by the subject (or where appropriate by the subject's legally authorized representative) for the duration of the study. All adverse events are coded and tabulated by body system, individual events within each body system and presented in descending frequency. Adverse events are also tabulated by severity and relationship to the study medication. Serious or potentially serious adverse events are summarised separately.

The following clinical laboratory tests are performed : (a) Hematology Panel including haemoglobin, hematocrit, erythrocytes, mean corpuscular volume (MCV), mean corpuscular haemoglobin mass (MCH), mean corpuscular haemoglobin concentration (MCHC), leukocytes (total WBC and automated differential counts), platelet count; (b) Chemistry Panel including gamma glutamyl transpeptidase (yGT), alanine aminotransferase (ALT), aspartate aminotransferase (AST), alkaline phosphatase, LDH, creatinine, uric acid, glucose, total bilirubin, total protein, albumin, cholesterol, triglycerides, urea, sodium, potassium, calcium, chloride and (c) Urinalysis including glucose, protein, blood, bicarbonate, citrate, pH. If abnormal protein or blood values are found, a microscopic inspection will be performed. Any clinically significant abnormalities persisting at the end of the study are followed until resolution, or until reaching a clinically stable endpoint.

A subject is considered as having completed the study if he/she has completed all three study days of the treatment phase. Subjects who are withdrawn from the study for any reason before completion of this phase are not considered to have completed.

Subject participation may be terminated prior to completing the treatment phase for any of the following reasons: (a) Adverse Event; (b) Subject choice; (c) Lost to follow-up, (d) Other. When a subject withdraws prior to completing the study, the reason for withdrawal is documented on the CRFs and in the source document. Study drug assigned to the withdrawn subject is not assigned to another subject. Subjects who withdraw prior to completing all scheduled assessments on study day 2 are replaced.

Efficacy Criteria:

The photosensitivity range is evaluated from 21-channel EEG recordings made during IPS sessions performed at the screening visit, shortly before study drug administration and at hourly intervals up to 8 hours post-dose on Days 1, 2 and 3. Mood is determined using the Profile of Mood States (POMS) instrument administered within 1 hour prior to study drug administration and at 1, 3 and 6 hours post-dose (after photosensitivity assessment and pharmacokinetic blood sampling have been performed) on Days 1, 2 and 3.

Complete suppression or 3 point reduction in the IPS sensitivity range in 2 out of 3 subjects is considered valid evidence of antiepileptic activity of test compound (i.e. a compound of formula (I)) at the dose level(s) at which this occurs. Failure to find a dose level at which either of the above criteria is met in at least one eye condition (open, closure, closed) is interpreted as insufficient effectiveness of the drug.

Efficacy Evaluations [Pharmacodynamics]:

The main objective of the study is to evaluate the acute antiepileptic effect of test compound (i.e. a compound of formula (I)). A secondary objective is to investigate the effect of test compound (i.e. a compound of formula (I)) on mood.

The statistical analysis of antiepileptic effect is based on the photosensitivity ranges provided by the 2 blinded investigators based on the EEG tracings, recorded during the IPS sessions. The photosensitivity ranges is expressed as lower and upper IPS-frequency limits (Hz) for each timepoint of assessment, and will be evaluated statistically as follows. Profiles of the photosensitivity ranges over all 3 study days are plotted for each individual. Individual percentage changes of the photosensitivity range area from dosing to 8 hours post-dose on day 2 as compared with the corresponding area of Day 1 are described. Individual percentage changes of the mean photosensitivity range post-dose of Day 2 from the photosensitivity range pre-dose of Day 2 are summarized. If for an individual a decrease in the photosensitivity range from pre-dose to post-dose of Day 2 of at least 3 points on the frequency scale (see Section 9.3) is observed then the reaction of this subject is interpreted as positive. These results are used for decision of dose changes in the dose finding procedure.

For the analysis of the secondary objective, mood, 6 factor scores is calculated as detailed in 9.3, Efficacy Evaluations: tension-anxiety, depression-dejection, anxiety-hostility, fatigue, vigor and confusion score. The results are shown as individual profiles of each factor over time.

Pharmacokinetic/Pharmacodynamic Analyses:

Two objectives are of interest, relationship of the antiepileptic effect to plasma levels, and interactions with pre-existing antiepileptic drugs. Both objectives are examined based on graphs showing profiles of plasma levels of test compound (i.e. a compound of formula (I)) and of eventual concomitant AEDs together with photosensitivity ranges over all 3 study days, one graph per subject.

The relation between change in photosensitivity range and test compound (i.e. a compound of formula (I)) plasma level are described as onset time, amount and duration of the antiepileptic reaction in relation to the time of the estimated peak blood level. Onset time of any antiepileptic reaction is where the change of the interpolated profile range reaches 50% of its maximal change. The duration is interpreted to end where the profile range again widens to more than 50% of its maximal change. The amount of the reaction is taken as the individual percentage change of the mean photosensitivity range post-dose of Day 2 from the photosensitivity range pre-dose of Day 2.

If any patients with concomitant AEDs are participating in the study, their graphs are compared with the graphs of non-AED patients, and sensitivity profiles and AEs for the two groups described to investigate any pharmacokinetic interactions.

### EXAMPLE 18

As a specific embodiment of an oral composition, 100 mg of the Compound #1 prepared as in Example 1 is formulated with sufficient finely divided lactose to provide a total amount of 580 to 590 mg to fill a size 0 hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A method for treating epilepsy or a related disorder, comprising administering to a subject in need thereof co-therapy with a therapeutically effective amount of one or more anti-epileptic or anti-convulsant agents and a therapeutically effective amount of compound of formula (I) 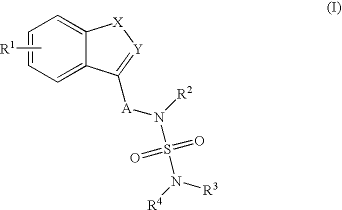
wherein
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of 0, N and S;
or a pharmaceutically acceptable salt thereof.

2. The method of claim 1 wherein
R¹ is selected from the group consisting of hydrogen, halogen, trifluoromethyl, cyano and nitro;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen, methyl and ethyl;
or a pharmaceutically acceptable salt thereof.

3. The method of claim 2, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each independently selected from the group consisting of hydrogen and ethyl;
or a pharmaceutically acceptable salt thereof.

4. The method of claim 3, wherein
R¹ is selected from the group consisting of hydrogen, 5-chloro, 5-fluoro, 5-bromo, 4-bromo, 7-fluoro, 5-trifluoromethyl and 5-cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each hydrogen; alternatively R³ is hydrogen and R⁴ is ethyl;
or a pharmaceutically acceptable salt thereof.

5. The method of claim 1, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

6. The method of claim 5, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 6 membered, saturated or aromatic ring structure, optionally containing one to two additional heteroatoms independently selected from the group consisting of O, N and S;
or a pharmaceutically acceptable salt thereof.

7. The method of claim 6, wherein
R¹ is hydrogen;
X—Y is —S—CH—;
A is —CH₂—;
R² is hydrogen;
R³ and R⁴ are taken together with the nitrogen atom to which they are bound to form a 5 membered ring structure selected from the group consisting of pyrrolidinyl and imidazolyl;
or a pharmaceutically acceptable salt thereof.

8. The method of claim 2, wherein the compound of formula (I) is selected from the group consisting of
N-(benzo[b]thien-3-ylmethyl)-sulfamide;
N-[(5-chlorobenzo[b]thien-3-yl)methyl]-sulfamide;
N-(3-benzofuranylmethyl)-sulfamide;
N-[(5-fluorobenzo[b]thien-3-yl)methyl]-sulfamide;
N-(1-benzo[b]thien-3-ylethyl)-sulfamide;
N-(1-naphthalenylmethyl)-sulfamide;
N-[(2-methyl-3-benzofuranyl)methyl]-sulfamide;
N-[(5-bromobenzo[b]thien-3-yl)methyl]-sulfamide;
N-[(4-bromobenzo[b]thien-3-yl)methyl]-sulfamide;
N-[(7-fluorobenzo[b]thien-3-yl)methyl]-sulfamide;
N-[(1-methyl-1H-indol-3-yl)methyl]-sulfamide;
N-[(4-trifluoromethylbenzo[b]thien-3-yl)methyl]-sulfamide;
N-[(4-cyanobenzo[b]thien-3-yl)methyl]-sulfamide;
N-[(benzo[b]thien-3-yl)methyl]-sulfamoylpyrrolidine;
N-[(benzo[b]thien-3-yl)methyl]-N′-ethylsulfamide;
imidazole-1-sulfonic acid [(benzo[b]thien-3-yl)methyl]-amide;
and pharmaceutically acceptable salts thereof.

9. The method of claim 1, wherein the compound of formula (I) is selected from the group consisting of N-(benzo[b]thien-3-ylmethyl)-sulfamide; N-[(5-fluorobenzo[b]thien-3-yl)methyl]-sulfamide; and pharmaceutically acceptable salts thereof.

10. A method for treating epilepsy or a related disorder, comprising administering to a subject in need thereof, co-therapy with a therapeutically effective amount of one or more anti-epileptic or anti-convulsant agents and a therapeutically effective amount of N-(benzo[b]thien-3-ylmethyl)-sulfamide; N-[(5-fluorobenzo[b]thien-3-yl)methyl]-sulfamide or a pharmaceutically acceptable salt thereof.

11. The method of claim 1, wherein the disorder is epilepsy.

12. The method of claim 1, wherein the related disorder is essential tremor or restless limb syndrome.

13. The method of claim 10, wherein the disorder is epilepsy.

14. The method of claim 10, wherein the related disorder is essential tremor or restless limb syndrome.

15. The method of claim 1, wherein the anti-convulsant or anti-epileptic agent is selected from the group consisting of carbamazepine, clobazam, clonazepam, ethosuximide, felbamate, gabapentin, lamotrigine, levetiracetam, oxcarbazepine, phenobarbital, phenytoin, pregabalin, primidone, retigabine, rufinamide, talampanel, tiagabine, topiramate, valproate, vigabatrin, zonisamide, benzodiazepines, barbiturates and sedative hypnotics.

16. The method of claim 15, wherein the anti-convulsant or anti-epileptic agent is selected from the group consisting of carbamazepine, clobazam, clonazepam, ethosuximide, felbamate, gabapentin, lamotrigine, levetiracetam, oxcarbazepine, phenobarbital, phenytoin, pregabalin, primidone, retigabine, rufinamide, talampanel, tiagabine, topiramate, valproate, vigabatrin and zonisamide.

17. The method of claim 16, wherein the anti-convulsant or anti-epileptic agent is selected from the group consisting of carbamazepine, gabapentin, lamotrigine, levetiracetam, oxcarbazepine, phenytoin, pregabalin, valproate and topiramate.

18. The method of claim 10, wherein the anti-convulsant or anti-epileptic agent is selected from the group consisting of carbamazepine, clobazam, clonazepam, ethosuximide, felbamate, gabapentin, lamotrigine, levetiracetam, oxcarbazepine, phenobarbital, phenytoin, pregabalin, primidone, retigabine, rufinamide, talampanel, tiagabine, topiramate, valproate, vigabatrin, zonisamide, benzodiazepines, barbiturates and sedative hypnotics.

19. The method of claim 18, wherein the anti-convulsant or anti-epileptic agent is selected from the group consisting of carbamazepine, clobazam, clonazepam, ethosuximide, felbamate, gabapentin, lamotrigine, levetiracetam, oxcarbazepine, phenobarbital, phenytoin, pregabalin, primidone, retigabine, rufinamide, talampanel, tiagabine, topiramate, valproate, vigabatrin and zonisamide.

20. The method of claim 19, wherein the anti-convulsant or anti-epileptic agent is selected from the group consisting of carbamazepine, gabapentin, lamotrigine, levetiracetam, oxcarbazepine, phenytoin, pregabalin, valproate and topiramate.
